(19) United States
(12) Patent Application Publication
(10) Pub. No.: US 2018/0303982 A1
(43) Pub. Date: Oct. 25, 2018

(54) WOUND DRAIN

(71) Applicant: Integrated Healing Technologies, LLC, Franklin, TN (US)

(72) Inventors: Albert Rodewald, Franklin, TN (US); Robert Stephen Porter, Brentwood, TN (US); John Armocast, Warsaw, IN (US)

(21) Appl. No.: 15/960,264

(22) Filed: Apr. 23, 2018

Related U.S. Application Data
(60) Provisional application No. 62/488,743, filed on Apr. 22, 2017.

(51) Int. Cl. A61M 1/00

(52) U.S. Cl. CPC A61M 1/0086; A61M 1/0084

(57) ABSTRACT

Provided herein is a wound drain useful in negative pressure wound treatment therapy. The wound drain advantageously includes structural components that prevent the drain from collapsing under negative pressure and from ingesting foreign materials introduced into a wound cavity, such as gauze and sponges, which con obstruct exudate flow through the drain. The wound drain may further comprise a dual lumen configuration such that it advantageously can be used in for positive pressure applications, or for combined negative and positive pressure treatments.

### CROSS-REFERENCE TO RELATED APPLICATIONS

This application claims benefit of the following U.S. Provisional Patent Application No. 62/488,743 filed on Apr. 22, 2017, which is hereby incorporated by referenced in its entirety.

### TECHNICAL FIELD

The present disclosure relates to a wound drain for use in wound care treatment, particularly negative pressure wound treatment.

### BACKGROUND OF THE INVENTION

Chronic and acute wounds, including pressure ulcers, diabetic wounds, and burns, present significant challenges to the health care industry. Patient care providers are actively seeking methods, devices, and systems for treating such wounds at a lower cost and with greater efficacy.

Conventional treatments for chronic wounds frequently include negative pressure therapy and/or hyperbaric oxygen therapy.

Negative pressure therapy is the controlled application of sub-atmospheric pressure to a wound using a therapy unit, such as a vacuum or suction device, to expose the wound to negative pressure to help promote wound healing. The wound is typically covered to facilitate the application of negative pressure and suction at the wound area. Various types of resilient, open cell foam surface dressings are typically sealed within an adhesive drape to provide the sub-atmospheric pressure at the wound site. Exudates are drained from the wound site and typically directed to a canister that stores the fluids and/or infectious material until properly disposed. Negative pressure wound therapy is often prescribed for chronic and acute wound types such as diabetic wounds, pressure ulcers, abdominal wounds, trauma wounds, various burns, flaps and grafts. However, negative pressure therapy may be less effective on patients having vascular disorders, such as diabetes, because negative pressure therapy can create a hypoxic environment at the wound. In current hospital settings, portable vacuum pumps are often rented or purchased for the purpose of providing negative pressure therapy. This can significantly increase the cumulative costs of providing wound care.

Hyperbaric oxygen therapy is the controlled application of oxygen to a wound at greater-than-atmospheric pressure(s). Oxygen is typically required for all new cell growth. Chronic or non-healing wounds tend to exhibit low oxygen tensions, or tend to be ischemic. A wound can become dormant if the amount of poorly oxygenated wound tissue reaches a critical mass. In this state, the body may no longer recognize the need to heal the affected area, thereby exacerbating the lack of oxygen in the wound and impairing healing of the wound by the body. Oxygen therapy is particularly useful for patients with poor circulation. The oxygen helps to kill bacteria and when applied to an open wound at a hyperbaric level, the oxygen is dissolved into the wound and absorbed by the surface wound tissue. The cells of the wound tissue that absorb the oxygen will begin metabolic activity in response to the increased oxygen tension. When the oxygen source is removed, the previously active cells request more oxygen from the body. The body responds by beginning to form new blood cells, and thus, starting the healing process. Accordingly, when delivered to a wound site under hyperbaric conditions, oxygen may act as a primary wound treatment fluid

More recently, wound care systems and methods have been developed that combine negative and positive pressure (e.g. hyperbaric oxygen) wound treatment therapies. Such methods and systems are described, for example, in U.S. Pat. No. 7,648,488, issued Jan. 19, 2010; U.S. Pat. No. 8,357,130, issued Jan. 22, 2013; U.S. Patent Publication No. 2008/0140029; and U.S. Patent Publication No. 2010/0121287.

Nevertheless, there is a need for wound drains that are particularly suited for negative pressure therapy or combined negative and positive pressure therapy. More particularly, there is a need for wound drains that have a structure that prevents collapse under negative pressure and avoids ingesting of foreign materials during therapy. The present disclosure addresses these needs.

### BRIEF SUMMARY OF THE INVENTION

The present disclosure provides a wound drain for a wound cavity, the drain comprising: a dome having a chamber wall defining an internal dome chamber with an open bottom; an integral peripheral flange extending radially from the dome; a port connection extending from the dome having a passage therethrough in open communication with the dome chamber; and a support structure seated within the dome chamber for preventing foreign object within the wound cavity from migrating into the dome chamber while allowing fluid flow from the wound cavity through the dome chamber and passage.

In certain embodiments, the support structure is a flat screen affixed to the flange and underlying the dome chamber. In other embodiments, the support structure is a grate having a plurality of apertures therethrough. In still other embodiments, the support structure is plurality of arcuate ribs.

In some embodiments, the chamber wall includes an annular lip extending internally into the dome chamber adjacent the flange to form an annular seat within the dome chamber, the support structure is a flat screen or a grate restrictively fitted within the seat.

In further embodiments, the dome and flange are composed of a pliable material and the support structure is composed of a pliable material more rigid than the material of the dome and flange.

In some embodiments, the dome comprises a dual lumen, wherein first and second lumens are in fluid communication with the interior of the dome

### BRIEF DESCRIPTION OF THE SEVERAL VIEWS OF THE DRAWINGS

FIG. 1 is a perspective view of an embodiment of the wound drain.

FIG. 2 is another perspective view of the wound drain of FIG. 1 showing internal features in shadow.

FIG. 3 is a side sectional view of the wound drain of FIG. 1.

FIG. 4 is an exploded perspective view of another embodiment of the wound drain of this invention.

FIG. 5 is a side sectional view of a third embodiment of the wound drain of this invention.

FIG. 6A depicts a side view of another embodiment of the wound drain. FIG. 6B is a cross sectional view of the wound drain depicted in FIG. 6A.

FIGS. 7A to 7C depict several views of an embodiment of a wound drain having a dual lumen configuration.

FIGS. 8A and 8B depict a grate useful in the wound drains of the present disclosure.

FIGS. 9A and 9B depict yet another embodiment of a wound drain according to the present disclosure.

### DETAILED DESCRIPTION OF THE INVENTION

The wound drain of the present disclosure advantageously incorporates structural components that prevent the drain from collapsing under negative pressure and from ingesting foreign materials introduced into the wound cavity, such as gauze and sponges, which can obstruct exudate flow through the drain. The wound drains are used in a conventional manner, being placed over wound sites and secured by an occlusive dressing hermetically sealing the wound site. The wound drains can be employed, for example, whenever a controlled distribution and/or collection of fluid(s) is desired. The wound drains facilitate negative pressure wound treatment, but may in other embodiments be adapted for use with combined negative and positive pressure (atmospheric or above) wound treatment.

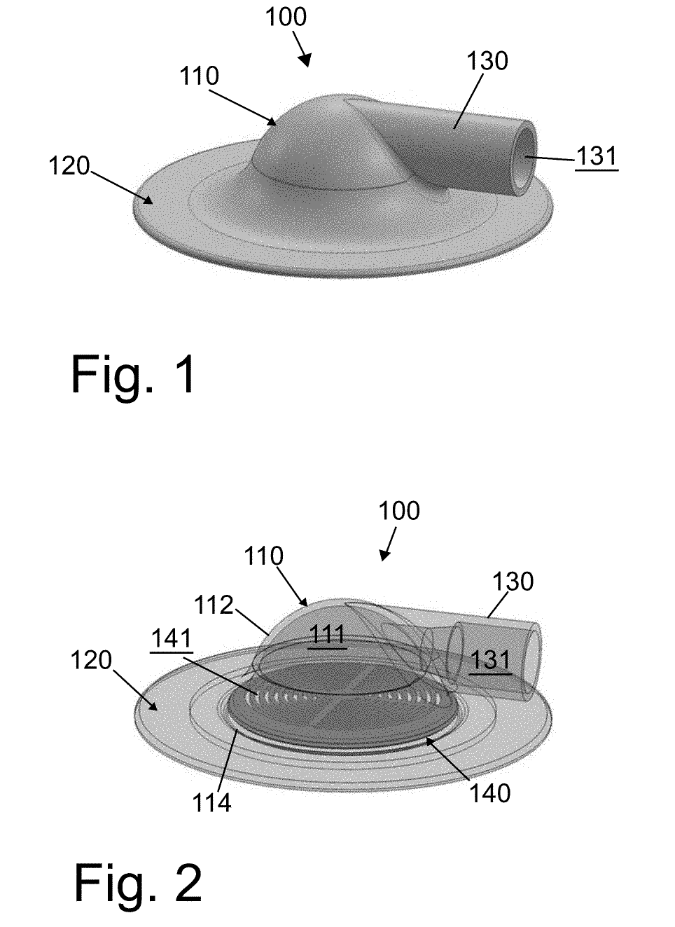

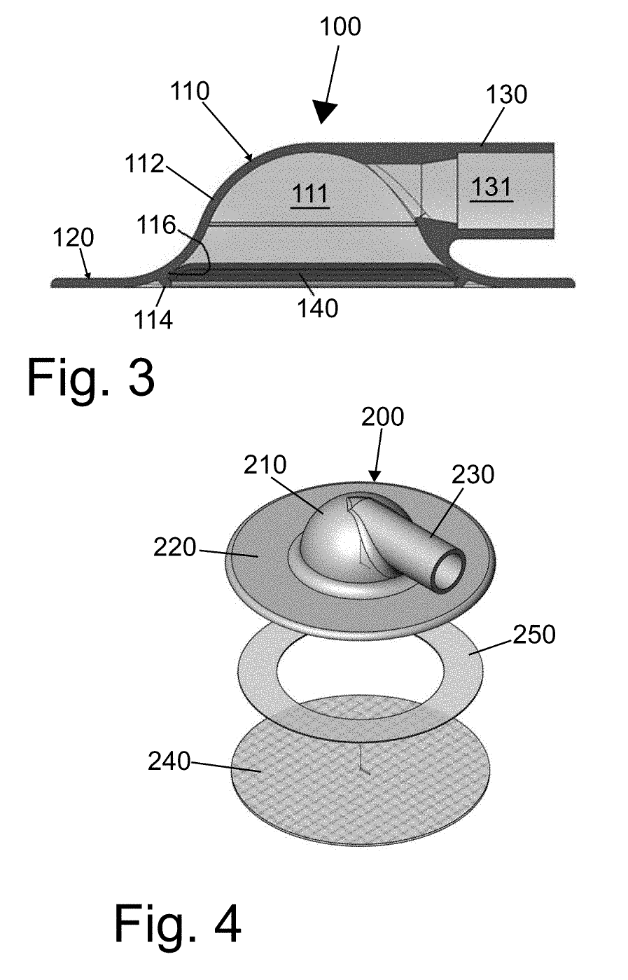

Referring now to the drawings, FIGS. 1-3 illustrate an embodiment of the wound drain, which is designated generally as reference numeral 100. Wound drain 100 is configured to have a central semi-spherical dome 110 integrally rising from a flat annular flange 120 extending radially from the dome. Dome 110 has a dome wall 112 defining an interior dome chamber 111 with an open bottom. Flange 120 transitions radially outward from dome wall 112 at the bottom of dome 110. Dome 110 also has an integral annular lip 114 that extends inward from dome wall 112 at the bottom of dome chamber 111 near the transition to flange 120. Dome 110 may also has a plurality of radial support ribs (not shown) extending into dome chamber 111.

Dome 110 also has a port connection 130 integrally formed therewith. Port connection 130 defines a fluid passage 131 in open communication with the dome chamber 111. Port connection 130 is configured to receive one or more fluid lines or lumens (not shown). The lumens are operatively connected to the negative pressure supply and/or drain line so that the negative pressure draws exudate into fluid passage 131 through dome interior 111.

Wound drain 100 is formed, molded or otherwise made of a pliable material, suitable for surgical and medical use. The pliable material may be a material suitable for medical purposes, such as a silicon rubber or Arkema Pebax® polymer. The pliable construction allows the drain to conform and contort so that the flange lies flat against the patient's skin, even over, around and into body creases and folds.

Wound drain 100 also includes a structural support or screen 140. As shown, screen 140 is configured as a flat circular disc having a plurality of concentric arcuate openings or slits 141 formed in a segmented quadrant pattern. In other embodiments, the openings or slits in the screen may take other shapes and configurations. Slits 141 allow exudate and other fluids to pass from the wound cavity into dome chamber 111, while preventing gauze and sponges from being ingested into the dome. The openings or slits are configured and dimensioned to provide sufficient area to allow fluid flow across the bottom of dome 110, but are small enough to prevent foreign materials from migrating into and clogging fluid passage 131 of port connection 130.

Screen 140 is restrictively seated at the bottom of dome 100 between an annular lip 114 and dome wall 112. The conjunction between lip 114 and dome wall 112 creates a screen seat 116 which holds screen 140 at the bottom of dome chamber 111 in a “press-fit” type connection. In certain embodiments, suitable adhesives may be added at screen seat 116 to further join and secure screen 140 within dome 110. Screen 140 is typically formed, molded or otherwise made of a pliable material, suitable for surgical and medical use, but also more rigid than the material of dome 110 and flange 120. The screen material is selected so that screen 140 when seated within dome 110 provides sufficient structural integrity to hold the dome wall upright and prevent dome wall 112 from deforming and collapsing under negative pressure. The dome shape has a generous radius to allow for more pliability and reduced pressure points on the wound.

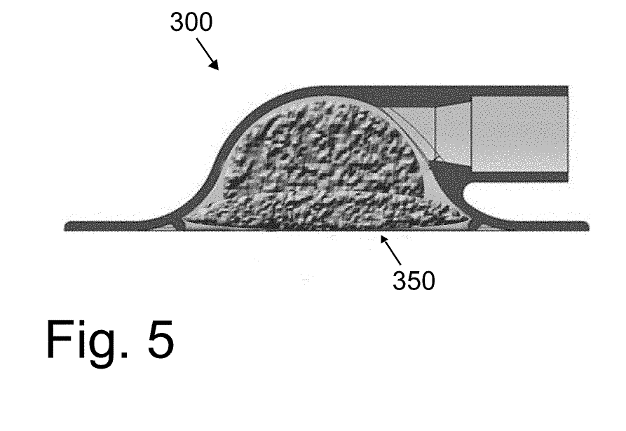

FIGS. 4 and 5 illustrate two alternative embodiments of the wound drain, identified respectively as reference numerals 200 and 300. Each wound drain 200 and 300 is identical in configuration and composition of drain 100 above, except that they employ a different structural component for preventing the drain from collapsing under negative pressure and from ingesting foreign materials introduced into the wound cavity, such as gauze and sponges, both of which can obstruct exudate flow through the drain. Wound drain 200 includes a dome 210, port connection 230 and a flat screen 240 that is affixed to the bottom of flange 220 covering substantially the entire surface area of drain 200. As with screen 140 above, screen 240 is typically formed, molded or otherwise made of a pliable material, suitable for surgical and medical use. More importantly, screen 240 is made of a material that is more rigid than the material of the dome and flange of drain 200. Screen 240 is affixed to the bottom surface of flange 220 by adhesive layer 250. Turning to FIG. 5, wound drain 300 includes a porous insert 350 restrictively seated within the dome chamber. Insert 350 is composed of a fibrous or open-celled matrix of suitable materials that allows fluid flow therethrough, but lends internal structural support to the dome wall. Insert 350 is configured to restrictively seat within the dome chamber in a “press-fit” type connection similar to that of drain 100.

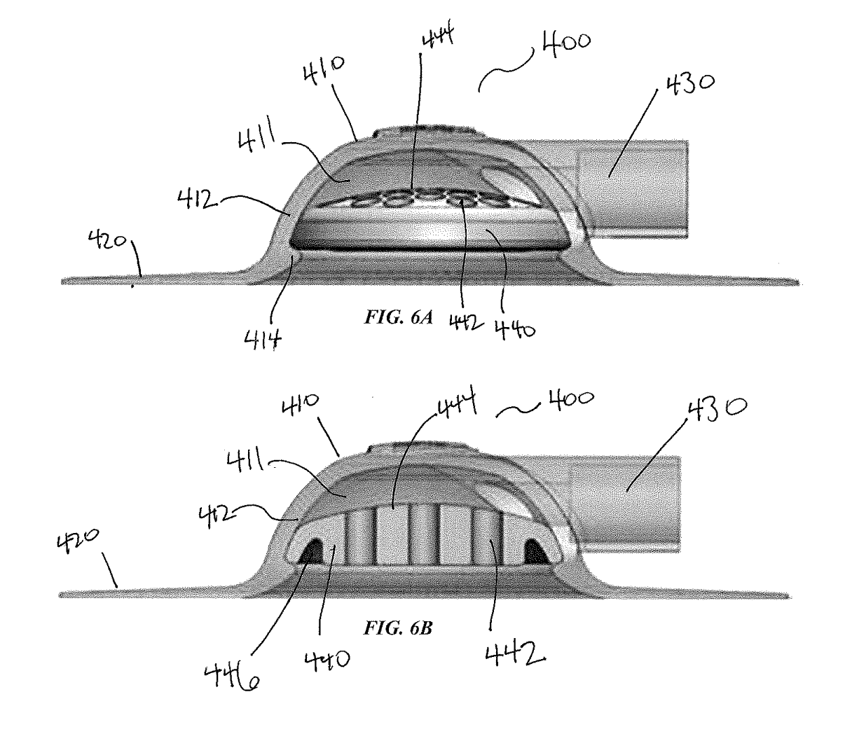

FIG. 6A depicts a side view of another embodiment of the wound drain of the present disclosure. Wound drain 400 has semi-spherical dome 410 integrally rising from flat annular flange 420 extending radially from the dome. Dome 410 has a dome wall 412 defining an interior dome chamber 411 with an open bottom. The dome further includes port connection 430 in fluid communication with dome interior 411. The dome 410 is shaped to have annular lip 414. Annular lip 414 holds a support structure, which in this embodiment is a grate 440. Grate 440 comprises a plurality of apertures providing fluid communication between the dome chamber 411 and a wound (not depicted). Grate 440 in some embodiments has a thickness that extends into the dome chamber. Grate 440 also may comprise a slightly domed or arcuate shape 444. FIG. 6B depicts a cross sectional side view of dome 400. Grate 440 may be shaped such that an annular cavity 446 extends upward towards dome 410. Wound drain 400 is formed, molded or otherwise made of a pliable material, suitable for surgical and medical use. The pliable material may be a material suitable for medical purposes, such as a silicon rubber or Arkema Pebax® polymer. The pliable construction allows the drain to conform and contort so that the flange lies flat against the patient's skin, even over, around and into body creases and folds. The support structure, such as grate 440, is made of a material that is more rigid than the dome material. It should be noted that the dome shape has a generous radius to allow for more pliability and reduced pressure points on the wound.

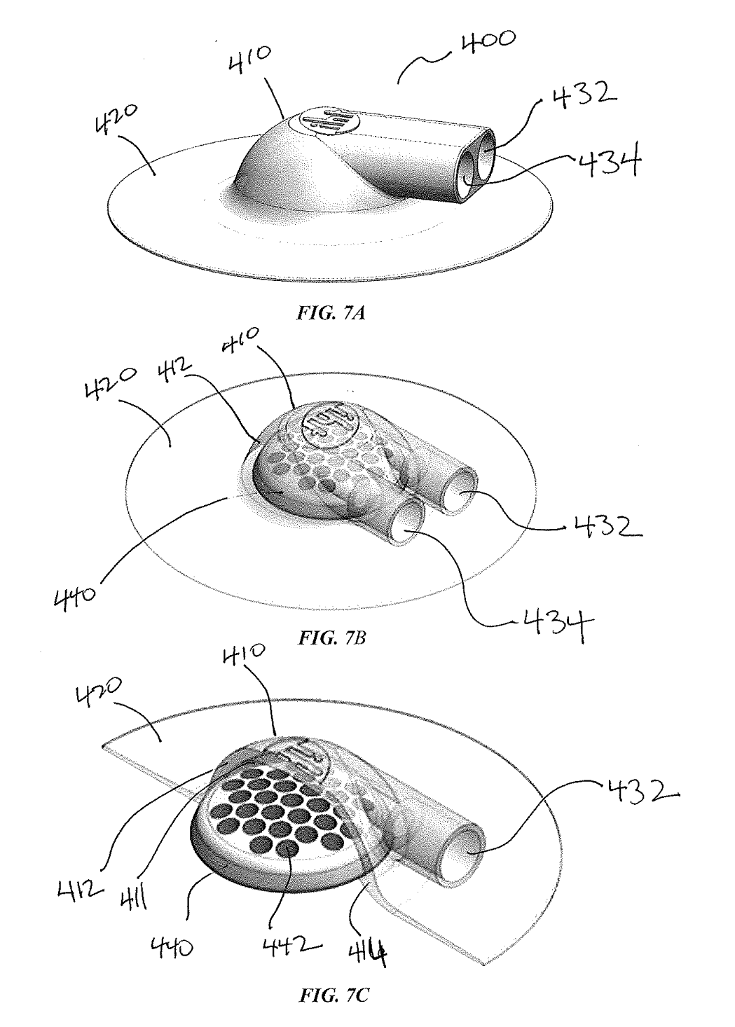

Turning to FIGS. 7A to 7C, an embodiment of a wound drain having a dual lumen is depicted. FIG. 7A depicts a perspective view of wound drain 400 having dome 410, flange 420 and first and second lumens 432 and 434, respectively, each in fluid communication with the dome interior 411. The first lumen provides connection to a drain line or negative pressure source (not depicted), while the second lumen advantageously provides a vent and/or an injection port for the introduction of fluids or gases. FIG. 7B is another perspective view depicting the interior of the dome 410 so that dome wall 412 and grate 440 can be seen. FIG. 7C is perspective partial cross sectional view of dome 400 depicting the apertures 442 of grate 440. FIG. 7C further shows dome interior chamber 411 and annular lip 414.

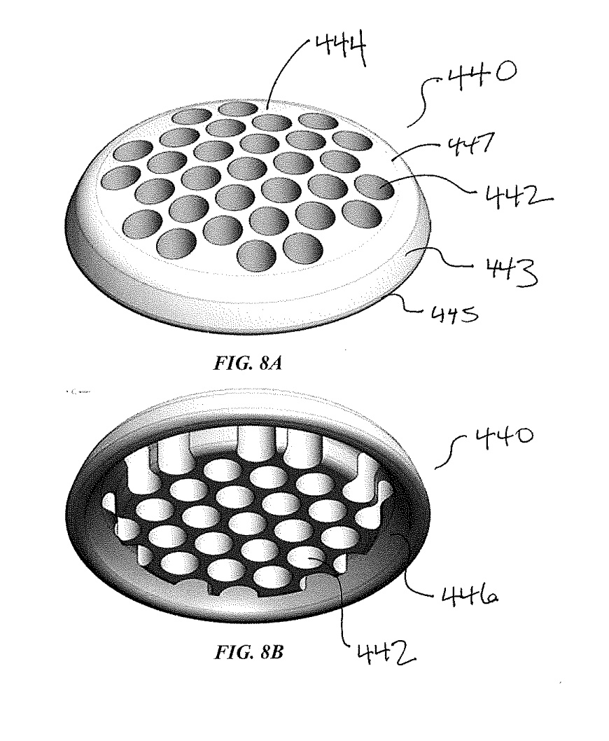

Turning to FIG. 8A, a top perspective view of grate 440 depicts a plurality of apertures 442, and comprises an arcuate shape 444. The apertures 442 may be of any size or shape suitable for allowing sufficient flow of wound exudate while preventing material from entering the lumen or lumens of the drain and also for manufacturing convenience. In this particular embodiment, apertures 442 are generally circular and arranged in a plurality of rows. The number of apertures can range from about 10 to about 100, about 10 to about 50, about 20 to about 50 or about 25 to about 35. Sidewall 443 of the grate generally curves inwardly, such that the radius of the bottom of the grate 445 is larger than the radius towards the top 447. FIG. 8B depicts a bottom perspective view of grate 440. Grate 440 may have a generally hollow annular ring 446.

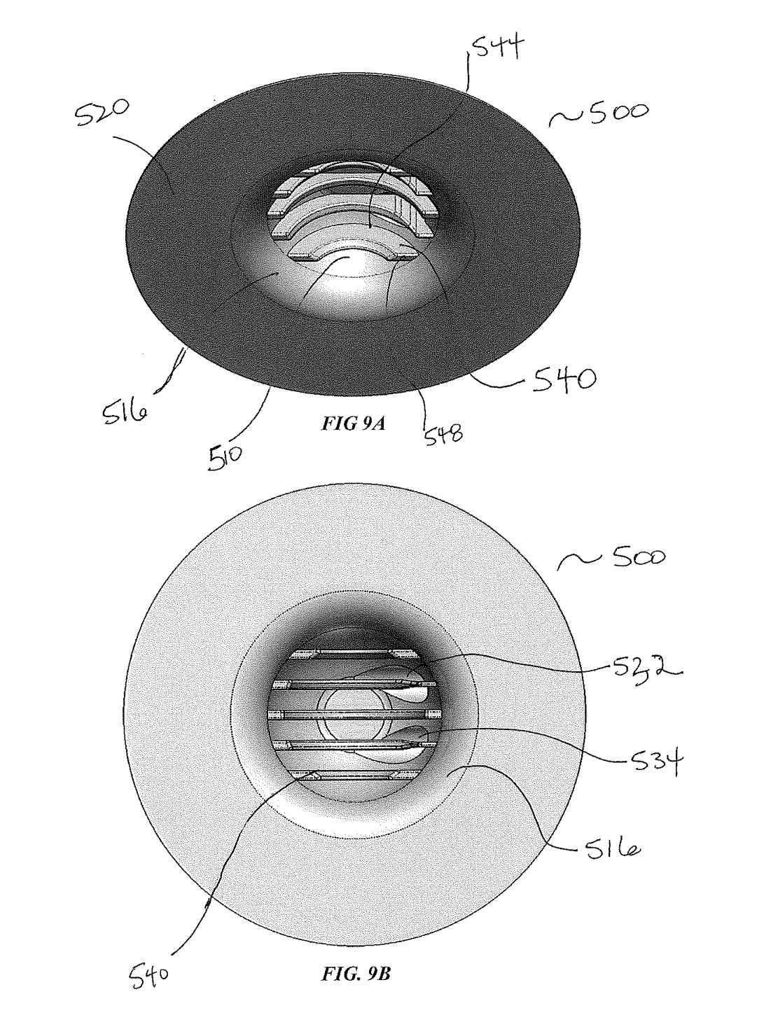

FIGS. 9A and 9B depict yet another embodiment of wound drain 500 having a dome 510 (depicted from inside the drain), flange 520 extending radially from the dome and curved portion 516 extending upwardly from the flange toward the dome 510. In this embodiment, drain 500 comprises a support structure of a plurality of arcuate ribs 540 integrally formed with curved portion 516. FIG. 9B depicts a perspective view from underneath the drain upward towards the dome. In this embodiment, dual lumens 532 and 534 are depicted.

Thus, although there have been described particular embodiments of the present invention of a new and useful wound drain it is not intended that such references be construed as limitations upon the scope of this invention except as set forth in the following claims.

What is claimed is:

1. A wound drain for a wound cavity, the drain comprising:
a dome having a chamber wall defining an internal dome chamber with an open bottom;
an integral peripheral flange extending radially from the dome;
a port connection extending from the dome having a passage therethrough in fluid communication with the dome chamber; and
a support structure within the dome chamber, the support structure comprising a plurality of openings for preventing foreign object within the wound cavity from migrating into the dome chamber while allowing fluid flow from the wound cavity through the dome chamber and passage.

2. The drain of claim 1 wherein the support structure is a flat screen affixed to the flange and underlying the dome chamber.

3. The drain of claim 1, wherein the support structure is a grate comprising a plurality of apertures.

4. The drain of claim 3, wherein the apertures comprise a circular shape.

5. The drain of claim 1, wherein the support structure comprises a plurality of arcuate ribs.

6. The drain of claim 1 wherein the chamber wall includes an annular lip extending internally into the dome chamber adjacent the flange to form an annular seat within the dome chamber.

7. The drain of claim 6, wherein the support structure comprises a flat screen fitted within the seat.

8. The drain of claim 6, wherein the support structure comprises a grate having a plurality of apertures fitted within the seat.

9. The drain of claim 1 wherein the dome and flange are composed of a pliable material and the support structure is composed of a pliable material that more rigid than the material of the dome and flange.

10. The drain of claim 1, further comprising a second port connection, the second port connection being in fluid communication with the dome chamber.

11. The drain of claim 10, wherein the second port connection is configured to provide a vent and/or an injection port for the introduction of fluid or gas into the dome chamber.

12. The drain of claim 1, wherein the support structure comprises a porous insert disposed within the dome chamber, the porous insert comprises a an open celled matrix material.
